(19) United States
(12) Patent Application Publication
(10) Pub. No.: US 2025/0349401 A1
(43) Pub. Date: Nov. 13, 2025

(54) SYSTEMS AND METHODS FOR STRESS DETECTION AND ACTION RESPONSE

(71) Applicant: WELLS FARGO BANK, N.A., San Francisco, CA (US)

(72) Inventors: Govinda Rajulu Nelluri, Hyderabad (IN); Vinay K. Pandey, Hyderabad (IN); Mutturaj Mundewadi, Bangalore (IN)

(21) Appl. No.: 19/090,210

(22) Filed: Mar. 25, 2025

Related U.S. Application Data
(60) Provisional application No. 63/644,697, filed on May 9, 2024.

(51) Int. Cl. G16H 10/60; A61B 5/00; A61B 5/16; G06N 20/20

(52) U.S. Cl. CPC G16H 10/60; A61B 5/165; A61B 5/7221; A61B 5/7267; G06N 20/20

(57) ABSTRACT

Systems and methods of stress detection of and action response via a set of operations including receiving a request from a user behavior data associated with the user. The operations further include converting the behavior data into one or more groups of feature vectors and applying the one or more groups of features vectors to one or more machine-learning models to generate a stress signal associated with the user. The operations include determining a stress response based on the stress signal and performing an action based in part on the request and the stress response.

### CROSS-REFERENCE TO RELATED APPLICATIONS

This application claims priority to U.S. Provisional Patent Application No. 63/644,697, filed on May 9, 2024, and entitled “SYSTEMS AND METHODS FOR STRESS DETECTION AND ACTION RESPONSE,” the entirety of which is hereby incorporated by reference herein.

### FIELD OF TECHNOLOGY

The present disclosure generally relates to automatic control of user interfaces, and more particularly to systems and methods for stress detection and response during a requested user interaction to manipulate user interface or computing resources.

### BACKGROUND

In authenticating user actions, such as a transaction between a user and a third party, the user's mental and emotional state may be a significant factor affecting such a transaction. For example, a user may perform a transaction under fear or duress that they otherwise would not have performed. Large percentages of regretted financial decisions are taken when a person is experiencing fear or agitation. Similarly, a user may be inebriated when making a request or attempting to perform a transaction leading to a later regretted action. Thus, impaired states such as fear or intoxication can lead to actions and decisions that users later wish were never authenticated and allowed. Current authentication mechanisms for reviewing user requests and transactions do not consider the emotional and behavioral state of the user, and instead look only towards traditional financial standards such as ISO-20022.

### SUMMARY

In an aspect, an example method includes receiving a request to perform an interaction associated with a user; receiving behavior data of the user; converting the behavior data into one or more groups of feature vectors; applying the one or more groups of feature vectors to one or more machine-learning models to generate a stress signal associated with the user; and performing an action based in part on the request and the stress signal associated with the user; determining a stress response based on the stress signal; and performing an action based in part on the request and the stress response.

In a further aspect, further example methods include receiving behavior data of the user from a variety of sensors including a camera feed, where the camera feed collects visual behavior data of the user to be processed by a trained visual-based machine-learning model.

In a further aspect, further example methods include receiving visual behavior data including facially recognized behavioral data, gesture-based behavioral data and environment-based behavior data to be processed by the trained visual-based machine-learning model.

In a further aspect, further example methods include receiving behavior data of the user from a variety of sensors including a keystroke sensor providing a keystroke feed, where the keystroke sensor collects keystroke behavior data of the user to be processed by a trained pattern recognition model; and from a biometric feed, where the biometric feed collects biometric behavior data to be processed by a trained biometric machine-learning model.

In a further aspect, further example methods include receiving keystroke behavior data including typing speed data, key-press frequency data, and/or typing error data.

In a further aspect, further example methods include receiving biometric behavior data including heart rate data and/or blood pressure data to be processed by the trained biometric machine-learning model.

In a further aspect, further example methods include determining the action to be performed based in part on comparing the stress signal with a confidence threshold.

In a further aspect, further example methods include performing actions comprising causing a user display to limit options otherwise available to the user in response to determining that the stress signal exceeds the confidence threshold.

In a further aspect, further example methods include adjusting the confidence threshold based in part on comparing the stress signal with location data of the user.

In a further aspect, further example methods include adjusting the confidence threshold based in part on receiving conflicting stress signals from the one or more machine-learning models.

In a further aspect, further example methods include adjusting the confidence threshold based in response to a user configuration.

The above methods can be implemented as computer-executable program instructions stored in a non-transitory, tangible computer-readable media and/or operating within a processor or other processing device and memory.

### BRIEF DESCRIPTION OF THE DRAWINGS

A full and enabling disclosure is set forth more particularly in the remainder of the specification. The specification makes reference to the following appended figures.

FIG. 1 illustrates an example system for determining an action based in part on a request by a user and a stress signal associated with the user, according to certain embodiments.

FIG. 2 illustrates a system for capturing behavioral data and encoding the behavior data into feature vectors, according to certain embodiments.

FIG. 3 illustrates a flowchart showing a method for determining an action based in part on a request by a user and a stress signal associated with the user, according to certain embodiments.

FIG. 4 illustrates an example data structure, or packet generated by the one or more machine-learning models for reception by an action decision module, according to certain embodiments.

FIG. 5 illustrates a block diagram for an example computing environment capable of executing the described systems and methods, according to certain embodiments.

### DETAILED DESCRIPTION

Reference will now be made in detail to various and alternative illustrative examples and to the accompanying drawings. Each example is provided by way of explanation, and not as a limitation. It will be apparent to those skilled in the art that modifications and variations can be made. For instance, features illustrated or described as part of one example may be used on another example to yield a still further example. Thus, it is intended that this disclosure include modifications and variations as come within the scope of the appended claims and their equivalents.

### Illustrative Embodiment of Stress-Response Action Determination

In one illustrative embodiment, a user may issue a request through an interface. The request can be a transaction request to debit money out of the user's financial account and deposit the information into another person's account. While the user is making the request, including for instance, as they open their phone into a banking app, or as they enter personal identification into an ATM, a variety of sensors may capture behavior data of the user and the surrounding environment of the user. The sensors may be cameras configured in a variety of locations such as on the user's phone or on an ATM able to acquire visual data of the user and the surrounding environment. In addition, or alternatively, the sensors can include the user's keystroke feeds, either by a mobile device or personal computer associated with the user. In the same or other embodiments still, the sensors can include biometric data sensors such as a smartwatch with a heart rate monitor worn by the user and paired with the user's smart phone or mobile device.

The behavior data of the user captured by the variety of sensors can be used as indicia of the user's mental state. The user's face captured by a camera sensor may display a look of contentment or fear. The user's gait, also acquired by the camera sensor, may indicate that the user is inebriated. The user's texts as tracked by a keystroke sensor may indicate the user is impaired (e.g., the user is pressing back space, delete, or the same key many times). The user's biometric patterns, as caught by the biometric sensor may indicate a high heart rate or high blood pressure as a further indicator of the user being in an agitated state. Detection modules tied to the sensors can receive the data fed in from the sensors and identify specific features to be stored and encoded for later use by predictive algorithms including machine-learning models.

The behavior data as caught and detected by the system may be auto encoded into respective feature vectors for input into trained machine-learning models. The respective autoencoding modules used may depend on the input channel of the behavior data. Textual autoencoding, as may be applied to the keystroke feed, can include a bag-of-words model, one-hot encoding, word-embedding, or other means of creating a representative vector from textual input. Similarly, visual-based auto-encoding methods may be used to transform camera feed data into representative vectors.

After being encoded into corresponding feature vectors, the behavior data, in feature vector form, may be input into respective machine-learning models including neural network models. The machine-learning model applied to each feature vector may be a structure specific to the feature vector. For example, camera-specific feature vector input may be fed into a convolutional neural network model such as the Facial Expression Recognition (“FER”) model. For keystroke-specific feature vectors, the machine-learning model can be a traditional classifier such as a Naive Bayes or Support Vector machine. More advanced techniques such as recurrent neural networks or long short-term memory networks (LSTM) may be used. For biometric specific feature vectors, the machine-learning model may include convolutional neural networks or recurrent neural networks to analyze physiological signals for emotion recognition tasks.

The method of training the machine-learning model may also be configured to the specific feature vector input. In some cases, the machine-learning model may already be pretrained, for instance, in the case of camera-specific feature vector input may include the pretrained library provided within the FER model.

Each of the one or more machine-learning models may output a stress signal with a confidence score in the emotional or mental state of the user. The confidence score may include multiple confidences indicating, for instance, that with 70% confidence that the user is in a state of fear and 30% in a state of anger. Any number of emotions and their respective confidences based on the processed feature vectors may be included within the output stress signal.

An action decision module may then receive a packet containing the user request, and the stress signal indicating a confidence in the user emotional and mental state, and other data such as user information data and location data. The action decision module may then determine an action to take in response to both the user request and the user's perceived emotional state based on the stress signal. The action may be communicated to a processor configured to perform the action. The action may consist of communicating to a third party that the user is under duress. For instance, if the action decision module receives an indication that the user is under duress with a 90% confidence, the action decision module may cause a processor to send an alert indication to the nearest police department. Other actions may consist of limiting the range of requestable information available to the user. For instance, if a user is indicated as being in a negative emotional state (e.g., sad, angry, confused) while requesting access to their bank account, the action decision module may signal to the requesting device to open the user's account, but to only show limited information related to the user's account, or that the user has available to them less money than what would normally be available had the user been identified in a non-distressed state.

### Example System for of Stress-Response Action Determination

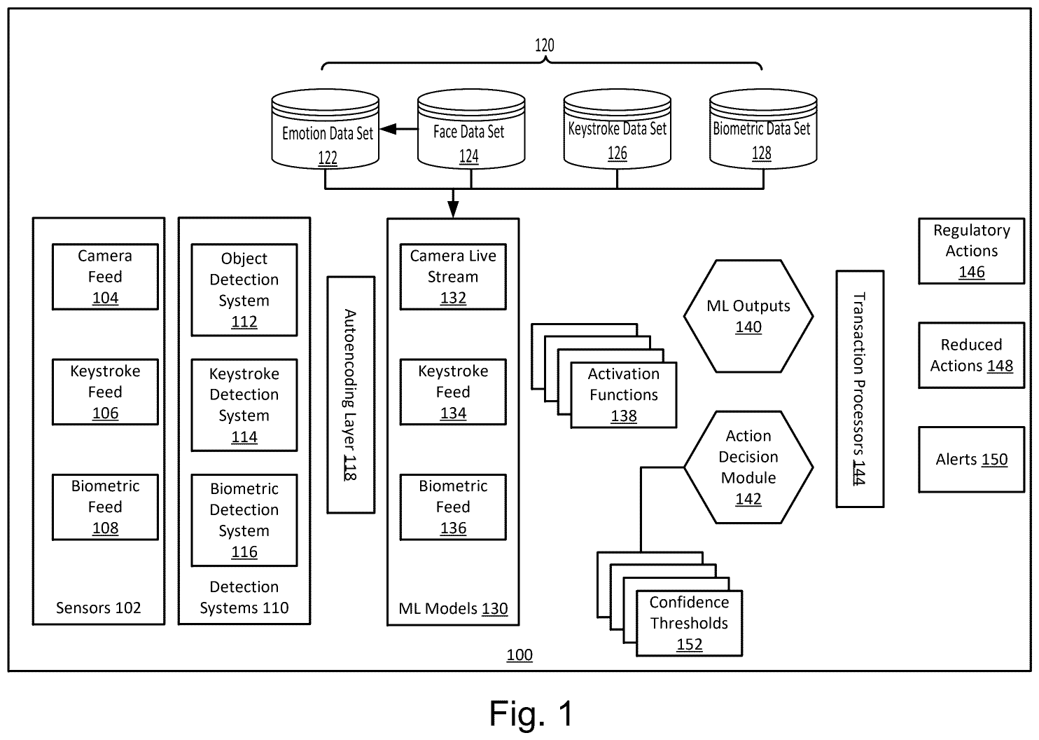

FIG. 1 illustrates an example system for determining an action based in part on a request by a user and a stress signal associated with the user, according to certain embodiments. The stress-responsive action determination system 100 includes a plurality of sensors 102 for capturing user behavior. The plurality of sensors 102 may include a camera feed 104, keystroke feed 106, and/or biometric feed 108 among other sensors. The plurality of sensors 102 may communicate with detection systems 110 capable of detecting and identifying relevant user behavior. For instance, the camera feed 104 that receives visual information related to a user behavior at the moment the user makes a request such as a transaction. The camera feed 104 captures the visual information of the user behavior where it is detected by an object detection system 112.

The user behavior data captured by the plurality of sensors 102 and detected by detection systems 110 (including for instance, object detection system 112, keystroke detection system 114, and biometric detection system 116) may then be preprocessed by a data preprocessor, and then converted into respective vectors, or feature vectors, at an autoencoding layer 118. The autoencoding layer 118 may include a variety of modules capable of transforming the user behavior data into feature vectors. For instance, different autoencoders will be applied to visual data compared to text based data inputs, which may further be different from biometric data inputs received from the sensor.

Once auto encoded into one or more feature vectors, the user behavior data may be input into respective machine-learning models 130 trained to identify associated mental states based on the user behavior data. In some instances, the machine-learning models 130 may comprise a variety of neural network architectures. For instance, a visual-based machine-learning model 132 may include a convolutional neural network trained on facial recognition data that may be applied to feature vectors specific camera feed data. Similarly, the pattern recognition model 134 and the biometric model 136 may be applied to data acquired from the keystroke detection system 114 and the biometric detection system 116 respectively. The machine-learning models 130 can generate one or more stress signals associated with the user. The stress signals may include a percent confidence that the user is in a specific mental state for instance fear or contentment.

The machine-learning models 130 may be trained on a variety of data sets 120 based on the machine-learning model to be trained. For instance, the visual-based machine-learning model 132 may be trained on a face data set 124 and/or an emotion data set 122 (e.g., a FER data set). The pattern recognition model 134 may be trained on typing attributes such as keystroke pattern data set 126, while the pattern recognition model 134 may be trained on biometric attributes included within the biometric data set 128. Typing attributes can include, for instance, key press duration, inter-key delay, typing speed, typing error-rate, typing pressure (e.g., as received via a touchscreen), typing patterns and rhythms, and the like. Biometric attributes used to train the biometric model 130 can include heart rate data, voice recognition data, respiration data, body temperature data, gait data and the like, where each may be capturable via biometric sensor associated with the user prior to and during initiation of the request.

Each of the machine-learning models 130 may be trained to analyze a variety of user behaviors in determining the state of the user while a request is being made. For example, the visual-based machine-learning model 132 may be trained to determine the mental and physical state of the user based on the camera feed 104 data. Factors that the visual-based machine-learning model 132 can be trained to analyze include facial characteristics of the user, such as the determining the user emotional state based on the angle of the user's eyes or mouth, whether the user's mouth is ajar, whether the user's eyes or blood shot, and other facial features indicative of the user's mental state. For instance, wide open eyes and a downturned mouth independently or in combination may be weighted towards an indication that the user is in a state of fear. Detected blood shot eyes and an open mouth may be weighted towards an indication that the user is intoxicated. An upturned mouth and upturned eyes may be individually weighed towards a stress signal confidence indicating the user is happy or content.

The visual-based machine-learning model 132 may also be trained to weigh gestures made by the user caught by the camera feed 104. Certain gestures may be weighed towards certain emotional states or associated responses. For instance, the user may make a thumbs down gesture as an SOS distress signal. The visual-based machine-learning model 132 may heavily weigh the detected gesture towards a prediction that the user is distressed.

The visual-based machine-learning model 132 may also be trained to analyze environment-based behavior data captured by the camera feed 104. For instance, the visual-based machine-learning model 132 may be trained to evaluate distances between the user initiating a request and the proximity of another person within the camera feed. The visual-based machine-learning model 132 may be trained to weigh a closer proximity of the other person as an indicator that the user is in distress. Other camera feed 104 data such as the weather, time of day, and relative brightness of the camera feed 104 may also be weighted. For instance, if the camera feed 104 detects that the feed is much darker, as in late at night, the visual-based machine-learning model 132 may weigh the environment towards stress signal confidence indicating distress.

The pattern recognition model 134 may be trained to determine the mental and physical state of the user based on the keystroke feed 106 of the user inputting the request. Keystroke feed 106 data can include not just keyboard entries, but other data gathered by physical acts of the user, such as moving of a mouse while the user is initiating a request by a computing device. Keystroke attributes that the pattern recognition model 134 can be trained to analyze include relative rate of typing of the user, or the frequency by which specific characters are pressed. For instance, a faster relative rate of typing may be weighed towards an indication that the user is under stress. Repetitive entry of a key, such as spacebar or backspace may be similarly weighed towards stress signal confidences of stress or that the user is in an incapacitated state. Entry of specific words and phrases may be used as an indicator of the user's mental state determined by the pattern recognition model 134. The pattern recognition model 134 may be trained to evaluate the rate at which the user is misspelling words or making grammatical errors, compared to the user's historic rates of making such mistakes to weigh a stress signal output towards specific mental states.

Aside from entry of data into keyboards, other data from the keystroke feed 106 may be input into the pattern recognition model 134 trained to predict the user's mental or emotional state. The pattern recognition model 134 may be trained to associate a high rate of mouse clicks or cursor movements with an agitated state of the user. When the keystroke feed 106 includes input from an ATM, the pattern recognition model 134 may be trained to weigh slower rates of keypad entry with specific mental states such as drowsiness.

The biometric model 136 may be trained to determine the mental and physical state of the user based on the biometric feed 108 of the user inputting the request. When the biometric feed 108 includes cardio data such as heart rate, and/or blood pressure (e.g., as acquired by photoplethysmography “PPG” sensors), the biometric model 136 may be trained to associate characteristics of the biometric feed 108 with specific states. For instance, lower heart rates may be weighed towards stress signal confidences indicating the user is happy, content, or relaxed. Elevated heart rates may be weighed towards stress signal confidences in fear, tension, or anger. The model may be trained to consider the age of the user and the user's overall health when receiving the user heart rate as input. The biometric model may be trained to evaluate changes in heart rates, such as rapid increase in heart rate prior to the request being initiated and weigh such increases towards a stress signal confidence that the user is experiencing anxiety.

The outputs 140 of each of the machine-learning models 130 may be placed in a larger machine-learning architecture used to make a determination on the stress signal associated with the user. For instance, the outputs 140 of the one or more machine-learning models 130 may include a data packet including behavior predictions to be fed into a set of activation functions 138 such as rectified linear units. The activation functions 138 may trigger based on values within the feature vectors output from the machine-learning models 130 to fully classify a final stress signal output. The final stress signal can be determined based on a combination of outputs from each activation function, where each activation function corresponds one of the machine-learning models 130. The stress signal output can include a confidence level in the emotional or mental state of the user as originally received by sensors 102 at the time before or during the requested action or transaction. For instance, the stress signal output can indicate with 70% confidence that a user is under duress at the time of a transaction. Alternatively, the stress signal output can output multiple confidences such as an indication that with 50% confidence the user is nervous and with 30% confidence indicate that the user is confused.

In some embodiments, the activation functions 138 units may receive packet data such as financial packet information. The financial packet information may include ISO-20022 compliant information including banking information such as account numbers, bank names, financial account status and other financial data.

The stress signal output can be transmitted to an action decision module 142 capable of determining an action in response to the received stress signal and additional packet information. The action decision module 142 can communicate with transaction processors 144 to perform an action 146-150 modifying or otherwise in response to the user request. For instance, the stress signal output received by the action decision module 142 may indicate with 80% confidence that the user submitting the request is inebriated. In response, the action decision module 142 may instruct a transaction processor 144 to prompt the user during the requested transaction to request additional authorization to finalize the transaction. The transaction processor 144 may be any processor able to interact with the user or a third party, for instance the processor used by the user to initiate the request.

Actions 146-150 include regulatory actions 146, reduced actions 148, and alerts 150, among other possible actions. Regulatory actions 146 can include logging the action requested by the user with the associated determined stress signal. In some examples, in response to heightened determined stress signals, and in response to a heightened request, regulatory actions 146 can include alerting relevant authorities of potential coercion as determined by the system. Reduced actions 148 can include modifying an interface to limit the user's ability to interact with a computing resource. For instance, if a stress signal exceeds a heightened threshold, indicative that the use is emotionally distressed with a heightened confidence, the action decision module 142 may signal to the requesting device to provide a second interface where read and access permissions are reduced. Alerts 150 may also be output, where the alerts can be output to the same interface initiating the request (i.e., to the requesting user) which indicate a perceived risk of performing an interaction. Additionally or alternatively, alerts can be transmitted to secondary devices such as devices associated with other users, including those within the requesting user's contacts, to provide an alert related to potential distress in request to perform an interaction.

The action decision module 142 may only take specified actions if the received stress signal exceeds a confidence threshold 154. The confidence threshold may be a minimum confidence value required by the received stress signal before the action decision module 142 instructs a transaction processor 144 to perform an action. The confidence threshold 154 can include multiple confidence thresholds where each confidence threshold is associated with a specific emotion. In some examples, the confidence threshold 154 can encompass confidences in multiple emotions, e.g., a confidence threshold of any perceived negative state such as fear, distress, and/or anger.

The confidence threshold 154 may be configured by a user or a system administrator. For instance, the user making requests may preconfigure the action decision module 142 to not take any actions unless a generated stress signal confidence indicating fear exceeds a confidence threshold 154 of 90%. A system administrator, such as a banking enterprise responsible for performing the requested action, may set a confidence threshold 154 of any perceived negative state requiring stress signals with 60% confidence in the negative state before causing an action.

In some examples, the action decision module 142 may determine a ranked set of actions based on one or more confidence thresholds 154. For instance, stress signals associated with fear exceeding a 60% confidence threshold 154 may cause the action decision module 142 to display reduced options on the user requesting device, while exceeding a 90% confidence threshold 154 may cause the action decision module 142 to contact authorities closest to the user's location.

The confidence threshold 154 may be a function of other data, for instance, the request data. In some examples, the severity of a user request may increase or decrease the confidence threshold 154 of a given emotion for a required action. Request severity may for example be the percentage withdrawal request from a user's bank account. A user requesting a withdrawal amounting to 1% of the user's bank account may require a higher confidence threshold 154 for an action to occur compared to a request for withdrawal of 50% of the user's bank account.

The confidence threshold 154 may be a function of location data that locates the user where the request is made. For instance, if a user is issuing a request from a hospital, the confidence threshold 154 for stress signals indicating distress may be raised compared to if the user was requesting from another location such as their home.

In some examples, the confidence threshold 154 may adjust based on input of one or more stress signals. For example, if conflicting stress signals each report high confidences, such as a stress signal indicating 60% confidence that the user is happy, and another stress signal indicating with 80% confidence that the user is upset, the confidence threshold 154 related to the anger-indicative stress signal may be raised to 90% before an action is to be performed to account for the conflicting stress signal confidences.

### Example Configurations Creating Encodings From Sensors

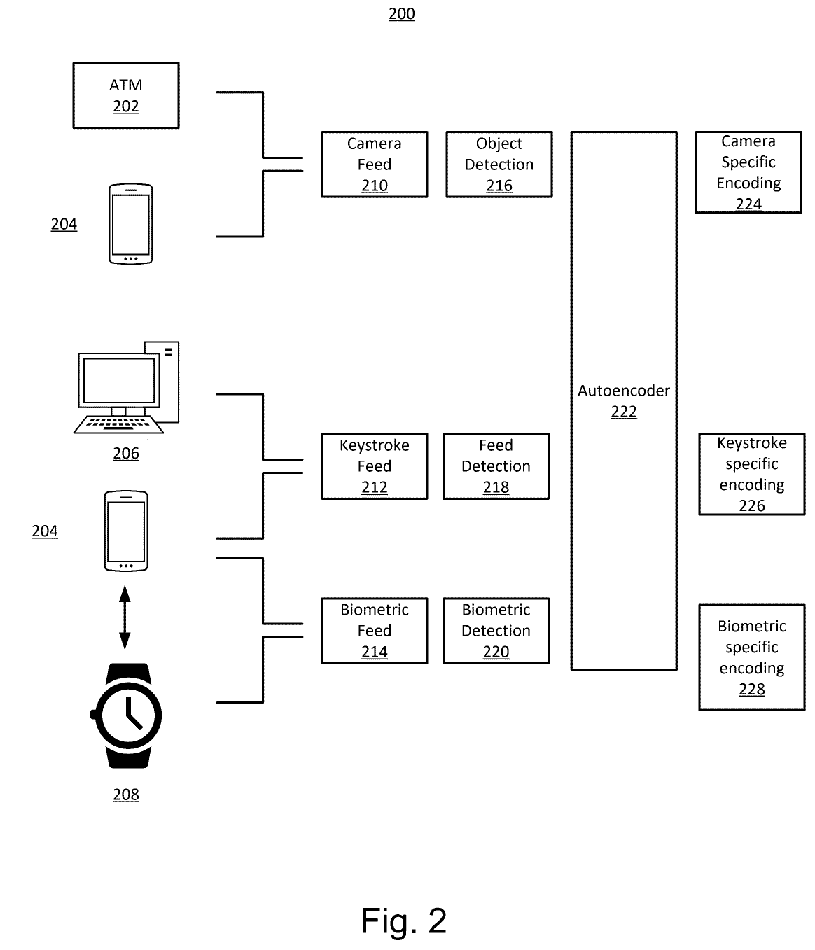

FIG. 2 illustrates a system for capturing behavioral data and encoding the behavior data into feature vectors according, according to certain embodiments. The system 200 includes data streams including camera feed 210, keystroke feed 212 and biometric feed 214 each capable of capturing behavior data for detection and auto encoding into respective feature vector encodings.

The camera feed 210 may include camera sensors included on an ATM or a mobile device 204. Other cameras capable of detecting a user while the user makes a request or performs a transaction may also feed into the camera feed 210. Camera monitors on a laptop or computing device may also be collected into the camera feed 210. Any combination of sensors 202-204 may be used for the camera feed 210.

The keystroke feed 212 may include any combination of keystroke tracking sensors capable of detecting a user's keystroke and digit press input prior to and while the user makes a request or performs a transaction. The user's personal computing device 206 or mobile device 204 may include a keystroke logger configured to communicate the keystroke feed 212.

The biometric feed 214 may similarly include any combination of sensors capable of monitoring a user during a user request or transaction. In one example, a smartwatch 208 or health monitoring device may communicate directly with the biometric feed 214 providing biometric behavioral data such as the user's heart-rate data or blood pressure data. In some examples, the smartwatch or other health monitoring device may be paired with the user's mobile device 204 and the mobile device 204 can communicate the biometric data to the biometric feed 214.

The mobile device 204 or other system providing user information or receiving user requests can further include a location sensor for providing location data of the user prior to and during the time the request is made. Location sensors can provide, for instance, Global Positioning System (“GPS”) coordinates locating the user at the time of the request.

Each feed 210-214 may also include a detection module 216-220 capable of capturing and preprocessing specific features from the respective feed. Object detection module 216 may filter out input into the camera feed 210 below a certain brightness or may save data storage by activating the camera feed 210 when the user is visible. Feed detection 218 may remove punctuation, convert text to lowercase, handle special characters and remove stop words. Biometric detection module 220 can collect data from the biometric feed 214 and preprocess the data by applying any necessary filtering techniques to remove noise or artifacts such heart rates outside a threshold boundary.

Autoencoder module 222 may include a variety of autoencoding programs specific to each data feed 210-216. The camera feed 210, generating visual image data, may apply image processing feature extraction techniques such as Local Binary Patterns, Scale-Invariant Feature Transforms, Speeded-Up Robust Features, or any other method of image data feature extraction. Feature extraction for keystroke feed 212 data may involve transforming techniques including bag-of-words, Term Frequency-Inverse Document Frequency, word embeddings such as Word2Vec or Global Vectors for Word representation, or any other natural language processing program. Feature extraction for the biometric feed 214 may include statistical measurements including calculating means or standard deviations, or frequency domain analysis such as spectral power or heart rate variability.

After input of the various feeds 210-214 into the autoencoder module 222, the autoencoder may generate respective feature vector encodings including camera specific encoding 224, keystroke specific encoding 226, and biometric specific encoding 228. Each feature vector encoding may be of fixed size. In some embodiments, not all feeds 210-214 may be active. In some cases, only the camera feed 210 may be present. In other embodiments only keystroke feed 212 and biometric feed 214 may present. It is to be appreciated that any combination of sensors and feeds may be provided for autoencoding behavior data gathered from a user.

### Example Operations for Stress-Responsive Actions

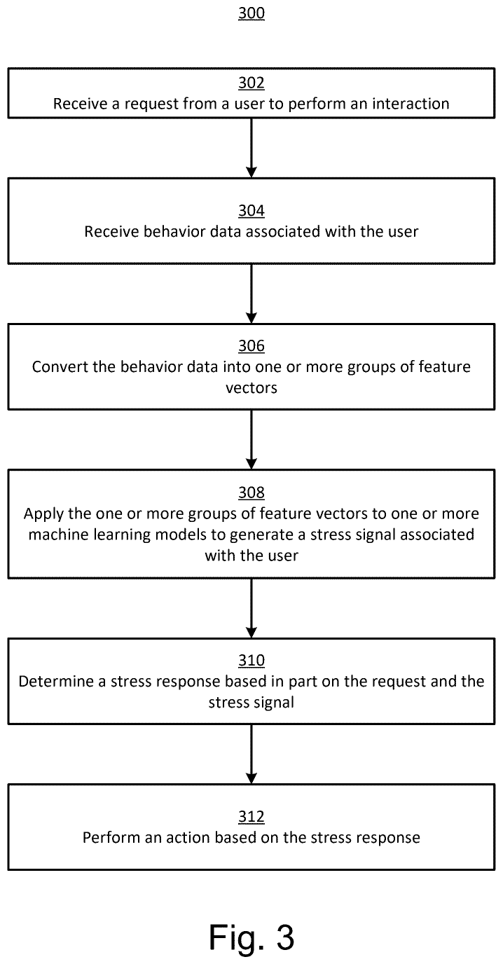

FIG. 3 illustrates a flowchart for a method 300 for determining an action based in part on a request by a user and a stress signal associated with the user, according to certain embodiments. In some examples, some of the steps in the flow chart of FIG. 3 are implemented in program code executed by a processor, for example, the processor in a general-purpose computer, mobile device, or server. In some examples, these steps are implemented by a group of processors. In some examples the steps shown in FIG. 3 are performed in a different order or one or more steps may be skipped. Alternatively, in some examples, additional steps not shown in FIG. 3 may be performed.

At block 302, the method 300 includes receiving a request from a user to perform an interaction. The request may include any variety of request to access a system or a resource. For instance, the request can include accessing an online account associated with the user, where the online account hosts a variety of resources associated with the user. Additionally or alternatively, the request can include a request to perform a transaction. For instance, the user may request to debit an account associated with the user and transfer funds to the account of another. As another example, the request may be to upload a document to a user program.

At block 304, the method 300 includes receiving behavior data associated the user. The behavior data can be received from a plurality of sensors. The plurality of sensors may include any one or more of the sensors discussed above with respect to FIGS. 1 and 2. The behavior data of the user may include data acquired from camera feed 210, keystroke feed 212, and/or biometric feed 214. The behavior data may be preprocessed according to the discussion above in FIG. 2. One or more of the sensors may be used to provide behavior data. According to one example, a keystroke feed may actively input behavior data while a biometric feed does not. According to other examples, each of the three sensors and feeds described with respect to FIG. 3 may be active, providing a camera feed, keystroke feed, and biometric feed. The behavior data may be received contemporaneously with the request as received at block 302 (e.g., within a designated time period leading up to and including the request).

Additionally or alternatively, the behavior data associated with the user can be received from one or more databases or other data storage devices. For example, keystroke data associated with the user can first be retrieved from the keystroke feed 212 and associated sensors, then stored in a database for subsequent retrieval and processing by the stress stress-responsive action determination system 100. Baseline behaviors can also be established by comparing more recent behavior data, as retrieved from the sensors, against historical averages, as stored in the one or more databases storing user behavior information. Comparing the more recent, contemporaneous stress signals against the baseline behavioral averages can provide an additional means for providing greater accuracy in stress detection and response.

The behavior data may be initially received from a different device than the device receiving the request per block 302. The keystroke feed, biometric feed, and/or camera feed may be received by a mobile device associated with the user, while the request may be received by a separate device, such as an ATM. The behavior data can also be received from one or more databases. Each of the request and the behavior data may be uploaded to a central server, storing the stress-responsive action determination system 100, where the stress-responsive action determination system 100 can generate a stress signal and determine a response as discussed further below. In other examples, the device receiving the request per block 302, and the device receiving one or more components of the behavior data (e.g., the camera feed or the keystroke feed) may be the same device, such as a mobile device associated with the user.

At block 306, the method 300 includes converting the behavior data into one or more groups of feature vectors. Autoencoder module 222 may perform any of the autoencoding techniques discussed above with respect to FIG. 2 to convert the behavior data into respective feature vectors. The groups of feature vectors include, for instance, the camera specific encoding 224, the keystroke specific encoding 226, and the biometric specific encoding 228.

At block 308, the method 300 includes applying the one or more groups of feature vectors to one or more machine-learning models to generate a stress signal associated with the user. The one or more machine-learning models may be applied in parallel or sequentially. For instance, visual-based machine-learning models 132 such as a FER Model may be applied to the camera specific encoding 224. The pattern recognition model 134 may be applied to the keystroke specific encoding 226, and the biometric model 136 may be applied to the biometric specific encoding 228. After the machine-learning models 132-134 are applied, further machine-learning operations may be performed including taking the output of these models, for instance, stress signals associated with the user, and further transmit the stress signals through further layers in a neural network to reach a composite or stress signal. The composite stress signal may be the sum of each of the outputs of the models 132-136 applied through a neural network including activation functions 138 such as rectified linear units.

Stress signals generated by the machine-learning models may indicate the system confidence in the user experiencing one or more emotions. For instance, the visual-based machine-learning model 132 may be trained on a labeled training set where images of facial expressions and associated emotional labels are input into the visual-based machine-learning model 132. The data set may label images of faces with furled brows as upset or faces with upturned lips as happy. Any number of features or facial expressions may be labeled as associated with a stress signal confidence. The visual-based machine-learning model 132 may also be trained on gesture images where specific gestures are labeled as associated with fear or distress. For instance, an image with a person holding their thumb down or holding their throat may be labeled as an image indicating high distress. The visual-based machine-learning model 132, once then trained on the image training data and labeled images, can generate stress signal confidence scores after receiving camera specific encodings.

Similar training techniques may be applied to the pattern recognition model 134 and biometric model 136. Data sets applied to train the pattern recognition model 134 may associate different behaviors to be received from the keystroke feed with particular emotions. Rate of key input may be labeled as indicative of one or more states depending on the key and on the frequency. For instance, high rates of entering the delete key may be labeled as indicative that the user is agitated or stressed. High rates of entry of other keys such as exclamation marks may provide confidences that the user is in a positive or agitated state.

The biometric model 136 may be trained on labeled data sets as well, where different heart rates are labeled corresponding to different emotional states. For instance, a heart rate of 100 bpm may be labeled as increasing the confidence that a user is upset or agitated while decreasing the confidence that a user would be happy or relaxed. Heart rate data may include further labels to calibrate for age, health, and the user's resting heart rate.

The output stress signals of each of the models 132-136 may be combined and summed to output a composite stress signal. For instance, the stress signal output by visual-based machine-learning model 132 may indicate that the user is distressed with 70% confidence, while the biometric model indicates with 80% confidence that the user is relaxed owing to a lower heart rate. The stress-responsive action determination system 100 in response may generate stress signals indicating that with 35% confidence that the user is distressed and with 40% confidence that the user is relaxed. The stress-responsive action determination system 100 may be configured to attach different weights to different stress signals. For instance, stress signal confidences output by the visual-based machine-learning model 132 may be weighed more heavily than stress signal confidences output by the pattern recognition model 134 and the biometric model 136. The weights may be configured manually by a system administrator or other user, or the weights may be adjusted within a larger neural network structure trained to generate a composite stress signal that sums the outputs of each of models 132-136. Each stress signal generated by a machine-learning model, including the composite stress signal may be stored as a hash value or may be otherwise encrypted between each stage of data transmission between nodes within a larger neural network architecture, or as output to the action decision module 142.

During the data processing stage between the one or more machine-learning models, the user request and additional user data may be input into the models. Additional user data may include ISO 20022 compliant data including payment identification, initiating debtor, initiating party, and other financial information. User data may also include the geographic location of the user originating the request. The user information can be appended to the composite stress signal.

At block 310, the process includes determining a stress response based in part on the request and the stress signal. The action decision module can include programmable instructions for causing different responses to occur based on the request type and the determined stress signal. Different request types may have several thresholds for classified stress signals. As an example, one request type may be to withdraw funds from a user account. The size of the funds requested can determine the thresholds for causing different actions to occur. A higher proportion of funds requested may lower the threshold stress signal scores required to restrict an action. Other request types can include requesting to access a computing resource or environment (e.g., a social media account belonging to the user, or a work-account belonging to the user).

Actions determined by the action decision module can include outright denying access to the account (e.g., when a threshold confidence in fear or inebriation exceeds 95% or other heightened threshold), outputting warnings or requesting additional verification or agreement to continue (according to the stress signal indicating stress above an intermediate threshold), or no action (e.g., when a stress signal falls below any assigned threshold).

Other actions which can be performed can include causing the functionality of various interfaces to be modified. A first type of interactive user experience can be associated with a first threshold stress signal, while a second interactive experience can be displayed in response to a second determined stress signal exceeding a heightened threshold providing access to a limited or modified resource associated with the user. As an example, if the stress signal is under each threshold, indicative that the user is not in a distressed state, a first user interface can be displayed, where the first user interface provides general access and read permissions. If the stress signal exceeds a heightened threshold, indicative that the use is emotionally distressed with a heightened confidence, the action decision module may signal to the requesting device to provide a second interface where read and access permissions are reduced (i.e., where the interface only shows limited information related to the user's account, or limited options to interact with the user's account user). Additionally or alternatively, traditional menu options in the interface or computer resource may be restricted or otherwise indicated as unavailable.

At block 312, the process includes performing an action based on the stress response. After the stress response is determined by block 310, the stress-responsive action determination system 100 can cause the determined action to be performed on one or more devices associated with the user. Instructions can be transmitted from the action decision module to the device which initially received the user request, such as a mobile device. A user's request to access bank account information on a mobile device may be transmitted to the stress-responsive action determination system 100 where, in response, the stress-responsive action determination system 100 transmits a signal back to the mobile device causing the determined action to be performed, such as limiting or denying access to the account on the mobile device, or limiting features available on the mobile device. In other examples, the stress-responsive action determination system 100 can transmit signals to other devices, such as devices associated with identified contacts of the user, or relevant authorities. In such an example, the determined action can include alerting a contact that the user is in a distressed state, where the alert is transmitted to a device associated with the contact.

### Example Data Structure For Transmission to an Action Determination System

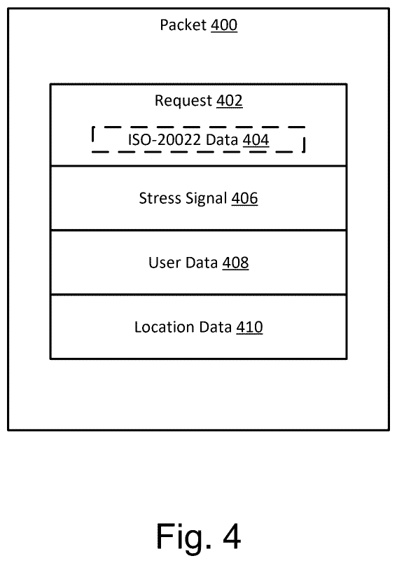

FIG. 4 illustrates an example data structure, or packet 400 generated by the one or more machine-learning models 130 for reception by action decision module 142, according to certain embodiments. In some examples, parts of the packet may be generated by the one or more machine-learning models 130 while other components of the data packet may be added to the packet 400 prior to input into the one or more machine-learning models. In other examples, components of the packet may be added after processing by the one or more machine-learning models 130.

The packet 400 may include a request 402. The request may be initiated by a user. The request can be for instance, a transaction request where the user requests to transfer money between the user's account and another person's account. When the request 402 relates to a financial transaction, the user data in the packet may contain ISO-20022 Compliant Data 404 or any other financial messaging standard compliant data. ISO-20022. ISO-20022 Compliant Data may include information such as a user's transaction ID, bank account ID, accounting number, and other financial accounting information.

The packet 400 may include stress signals 406. The stress signal 406, or behavior prediction, is a confidence value in the expected mental or emotional state of the user making the request. The stress signal may be a composite of individual stress signals generated through the machine-learning process by different machine-learning models. The stress signal may be the stress signal output of only the visual-based machine-learning model 132, where the stress signal is based on behavior data acquired only by the camera feed 104. Alternatively, the stress signal may be based on a combination of the stress signals output by the visual-based machine-learning model 132, the pattern recognition model 134, and/or the biometric model 136. The stress signal can contain one or multiple confidences in the mental and emotional state of the user. For instance, the stress signal may indicate that the user is content. The stress signal can indicate that the user, with 70% confidence, is under stress. The stress signal can indicate that the user, with 60% confidence that the user is angry and with 30% confidence that the user is confused.

The packet 400 may include user data 408. User data 408 can include any data indicating the user making the request. User data may include, for example, the user's IP address or a hash associated with the user in an enterprise system. The packet may also include location data 410. Location data 410 may include the coordinates of the user making the request while the request is being made.

The packet 400 may include one or more of the data 402-408 described above. For instance, location data 410 may or may not be included within each packet 400. The packet 400 and its constituent data may be stored and transmitted as a hash value, or otherwise encrypted as it is transferred across the stress-responsive action determination system 100. Once the packet is received by the action decision module 142, the action decision module 142 may decrypt the packet 400 for processing and determination of an action response.

### Example Procedures of the Action Decision Module

Once the action decision module 142 receives the packet 400, the action decision module 142 can determine a variety of actions in response to the user request and a confidence in the user state. The following examples are illustrative and non-limiting. In one example, action decision module 142 receives a confidence score indicating that the user is in distress (e.g., if the stress signal exceeds a confidence threshold 154 of 60% in emotional states of anger, fear, or depression, the action decision module 142 may determine that the user is not in a positive mental state. In response, the action decision module 142 can instruct a transaction processor to show the user account balance as a fraction of what the user actually possess in their account so as to prevent withdrawal actions. Alternatively, the action decision module 142 may instruct the transaction processor to show the full account balance of the user as greyed out on a user display and require additional actions by the user in order to access the full account.

In one example, the threshold stress signal confidence for determining a user action may vary based on the other data within the packet such as the content of request 402. For instance, if a user requests transfer over a threshold amount (e.g., greater than $1,000) from the user account, the action decision module 142 may reduce the confidence threshold 154 to take an action. In response, the action decision module 142 can flag such a request as suspicious activity requiring further authentication. The action decision module 142 may then evaluate the stress signal to determine the emotional state of the user. If the stress signal indicates with the user is in a state of fear with 60% confidence, the action decision module 142 module can instruct transaction processors 144 to inform relevant authorities (e.g., authorities located with a threshold distance of the user's location based on location data). If the user were instead requesting transfer of $100, and the user account indicates that the user possess $1,000,000, the action decision module 142 may require a stress signal confidence of 95% before taking an action.

In another example, the location data may be used to modify the confidence threshold 154 required to take an action. For instance, if the location data 410 indicates that the user is transmitting a request from a stress-designated location (e.g., a hospital, location of a natural disaster, or the like) the action decision module 142 may increase the confidence threshold 154 for a stress signal indicating distress from 70% to 90% before taking an action such as limiting access to the user's banking information.

### Example Computing Environment

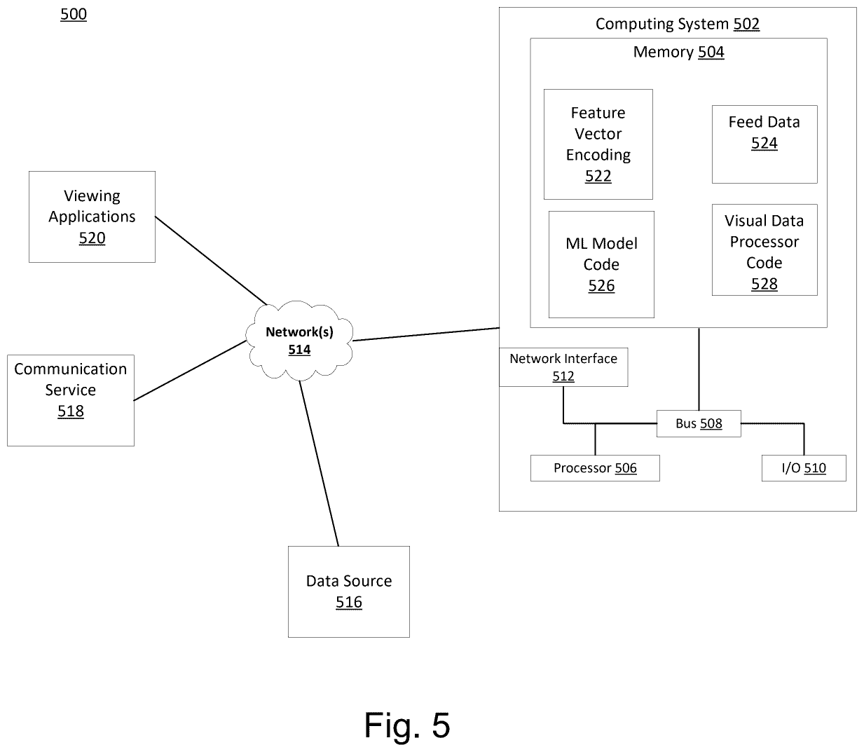

Any suitable computing system or group of computing systems can be used for performing the operations described herein. For example, FIG. 5 illustrates a block diagram for an example computing environment capable of executing the described systems and methods, according to certain embodiments. Components of computing system 502 may be used to execute functions called for by the stress-responsive action determination system 100 including its subsystems such as the one or more machine-learning models 130 and action decision module 142.

The depicted example of a computing environment 500 includes a computing system 502 which includes one or more processors 506 communicatively coupled to one or more memory devices 504. The processor 506 executes computer-executable program code or accesses information stored in the memory device 504. Examples of processor 506 include a microprocessor, an application-specific integrated circuit (“ASIC”), a field-programmable gate array (“FPGA”), or other suitable processing device. The processor 506 can include any number of processing devices, including one.

The memory device 504 includes any suitable non-transitory computer-readable medium for storing program code and data from a data source 516 such as the feed data 524 from the plurality of sensors 102 of embodiments discussed with respect to FIG. 1 and its related parameters including the camera specific, keystroke specific, and biometric specific feature vector encodings. Data processed and output by the one or more machine-learning models 526 may also be stored in memory device 504. The computer-readable medium can include any electronic, optical, magnetic, or other storage device capable of providing a processor with computer-readable instructions or other program code. Non-limiting examples of a computer-readable medium include a magnetic disk, a memory chip, a ROM, a RAM, an ASIC, optical storage, magnetic tape or other magnetic storage, or any other medium from which a processing device can read instructions. The instructions may include processor-specific instructions generated by a compiler or an interpreter from code written in any suitable computer-programming language, including, for example, C, C++, C#, Visual Basic, Java, Python, Perl, JavaScript, and ActionScript.

The computing system 502 may also include a number of external or internal devices such as input or output devices. The computing system 502 is shown with an input/output (“I/O”) interface 510 that can receive input from input devices or provide output to output devices. A bus 508 can also be included in the computing system 502. The bus 508 can communicatively couple one or more components of the computing system 502.

The computing system 502 executes program code that causes the processor 506 to perform one or more of the operations described above with respect to FIGS. 1-3. The program code includes operations related to, for example, receiving sensor data, processing the data, training machine-learning models, applying the data to the machine-learning models, and determining an action response. The program code may be resident in the memory device 504 or any suitable computer-readable medium and may be executed by the processor 506 or any other suitable processor. In some embodiments, the program code described above, the sensor input from streams 104-108, associated feature vector embeddings 224-228, and behavior prediction data are stored in the memory device 504, as depicted in FIG. 5. Memory 504 may also include a visual data processor code 528. In additional or alternative embodiments, the stream data 524, the feature vector encodings 522, the code and attributes underlying the machine-learning models 526, and other data provided in the stress-responsive action determination system 100 described above are stored in one or more memory devices accessible via a data network, such as a memory device accessible via a cloud service.

The computing system 502 depicted in FIG. 5 also includes at least one network interface 512. The network interface 512 includes any device or group of devices suitable for establishing a wired or wireless data connection to one or more data networks 514 such as viewing applications 520 including user interfaces. Non-limiting examples of the network interface 512 include an Ethernet network adapter, a modem, and/or the like. Remote communication services 518 are connected to the computing system 502 via network 512, and remote communication services 518 can perform some of the operations described herein, such as storing a set or subset of a data source 516. The computing system 502 is able to communicate with one or more of the remote communication services 518, the stress-responsive action determination system 100 and other data 516 using the network interface 512.

### Example Advantages of Systems and Methods for Stress-Response Action Determination

Certain aspects described herein provide improvements to authenticated computer environments over traditional authentication systems. Traditional authentication systems are directed towards entity verification using personal identification numbers, tokens, biometrics (e.g., fingerprints and facial recognition) prior to permitting access to a computing environment and associated computing resources. However, traditional authentication will fail to prevent unauthorized actions if an authenticated user initiating a request is doing so under duress, under another's volition, or if they are impaired by a mental state that clouds the user's judgment. Thus, techniques for emotionally validating user access to computing resources provides advantages over previous authentication techniques. Providing several different levels of access to computing resources provides a technical benefit to the computing environment. By causing a display associated with the computing resource to present different interfaces and interactive environments based on a user's determined stress level, such interfaces are improved in the particular manner in which information and resources are presented via the electronic device interfaces.

Certain aspects described herein also provide technical improvements in the means by which emotional authentication systems determine a user's emotional state prior to authorizing access to the computing environment. A combination of sensors including biometric sensors, camera feeds, and keystroke sensors may be arranged to simultaneously generate emotional classifications of users in-real time. The sensors may provide data to corresponding machine-learning models trained on respective data sets to provide more accurate, real-time classification of a user's stress level. Thus, the particular arrangement of sensors, and particular arrangement of processing data from each of the sensors (e.g., via respective machine learning models trained on associated attributes), allow for real-time emotional state determinations to be made to more effectively restrict and limit access to computing resources.

### General Considerations

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general-purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied-for example, blocks can be re-ordered, combined, and/or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art.

What is claimed is:

1. A method comprising:
receiving a request to perform an interaction associated with a user;
receiving behavior data associated with the user;
converting the behavior data into one or more groups of feature vectors;
applying the one or more groups of features vectors to one or more machine-learning models to generate a stress signal associated with the user;
determining a stress response based on the stress signal; and
performing an action based in part on the request and the stress response.

2. The method of claim 1, wherein the behavior data is received from a camera, and the one or more machine-learning models includes a trained, visual-based machine-learning model.

3. The method of claim 2, where the behavior data includes one or more of facially recognized behavioral data, gesture-based behavioral data, or environment-based behavior data.

4. The method of claim 1, wherein the behavior data is received from one or more of a keystroke sensor or a biometric sensor configured to collect biometric behavior data.

5. The method of claim 4, wherein the behavior data includes typing speed data, key-press frequency data, or typing error-rate data, and the one or more machine-learning models includes a pattern recognition model trained on typing attributes.

6. The method of claim 4, wherein the biometric behavior data includes heart-rate data or blood pressure data, and the one or more machine-learning models includes a biometric model trained on biometric attributes.

7. The method of claim 1, wherein performing an action based in part on the request and the stress signal includes:
determining, by the one or more machine-learning models, a stress signal confidence score;
comparing the stress signal confidence score to a confidence threshold; and
determining the action to be performed in response to determining the stress signal confidence score exceeds the confidence threshold.

8. The method of claim 7, where in the action comprises restricting access to one or more resources available to the user.

9. The method of claim 1, wherein the behavior data is received from a location sensor and the stress signal is based in part on location data associated with the user.

10. The method of claim 1, wherein the one or more machine-learning models includes a visual-based machine-learning model, a pattern recognition model, and a biometric model, wherein each of the one or more machine-learning models outputs to a respective activation function, and the stress signal is determined based on outputs of each respective activation function.

11. A system comprising:
a memory device; and
a processing device coupled to the memory device, the processing device configured to perform operations comprising:
receiving a request to perform an interaction associated with a user;
receiving behavior data associated with the user;
converting the behavior data into one or more groups of feature vectors;
applying the one or more groups of features vectors to one or more machine-learning models to generate a stress signal associated with the user;
determining a stress response based on the stress signal; and
performing an action based in part on the request and the stress response.

12. The system of claim 11, wherein the behavior data is received from a camera, and the one or more machine-learning models includes a trained, visual-based machine-learning model.

13. The system of claim 12, where the behavior data includes visual behavior data including one or more of facially recognized behavioral data, gesture-based behavioral data, or environment-based behavior data.

14. The system of claim 11, wherein the behavior data is received from one or more of a keystroke sensor configured to collect keystroke behavior data of the user, or a biometric sensor configured to collect biometric behavior data associated with the user.

15. The system of claim 14, wherein the behavior data includes typing speed data, key-press frequency data, or typing error-rate data, and the one or more machine-learning models includes a pattern recognition model trained on typing attributes.

16. The system of claim 14, wherein the biometric behavior data includes heart-rate data or blood pressure data, and the one or more machine-learning models includes a biometric model trained on biometric attributes.

17. The system of claim 11, wherein performing an action based in part on the request and the stress signal includes:
determining, by the one or more machine-learning models, a stress signal confidence score;
comparing the stress signal confidence score to a confidence threshold; and
determining the action to be performed in response to determining the stress signal confidence score exceeds the confidence threshold.

18. The system of claim 17, where in the action comprises restricting access to one or more resources available to the user.

19. The system of claim 11, wherein the behavior data is received from a location sensor and the stress signal is based in part on location data associated with the user.

20. A non-transitory computer readable medium storing executable instructions, which when executed by a processing device, cause the processing device to perform operations comprising:
receiving a request to perform an interaction associated with a user;
receiving, from one or more sensors, behavior data associated with the user;
converting the behavior data into one or more groups of feature vectors;
applying the one or more groups of features vectors to one or more machine-learning models to generate a stress signal associated with the user;
determining a stress response based on the stress signal; and
performing an action based in part on the request and the stress response.
